(19) United States
(12) Patent Application Publication
(10) Pub. No.: US 2009/0177035 A1
(43) Pub. Date: Jul. 9, 2009

(54) ENDOSCOPE INSTRUMENTS SYSTEMS AND METHODS FOR CLOSED CHEST EPICARDIAL ABLATION

(76) Inventor: Albert K. CHIN, Palo Alto, CA (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/347,112

(22) Filed: Dec. 31, 2008

Related U.S. Application Data
(60) Provisional application No. 61/018,876, filed on Jan. 3, 2008.

(51) Int. Cl. A61B 1/04; A61B 18/18

(52) U.S. Cl. 600/112; 600/109; 606/41

(57) ABSTRACT

Endoscopic surgical instruments, lens elements and methods of treating or ablating tissue such as epicardial surfaces of cardiac tissue. An endoscopic surgical instrument includes an elongate shaft, a lens attached to the distal end of the shaft, and a coupling element extending from or attached to the lens. The distal end of the lens can protrude through the coupling element so that an ablation element, such as a flexible microwave ablation element, held by the coupling element is in the line of sight of the lens. Embodiments can be used to selectively ablate epicardial surfaces to treat atrial fibrillation and form more complete lesions around pulmonary veins without severing or penetrating a pericardial reflection near the superior vena cava.

This application claims priority to Provisional Application Ser. No. 61/018,876 entitled “Endoscope Instruments Systems and Methods for Closed Chest Epicardial Ablation” filed Jan. 3, 2008, which is incorporated herein by reference.

### FIELD OF THE INVENTION

The present invention relates to surgical devices and methods for ablation of epicardial surfaces of cardiac tissue.

### BACKGROUND

Atrial fibrillation is a condition in which upper chambers of the heart beat rapidly and irregularly. One known manner of treating atrial fibrillation is to administer drugs in order to maintain normal sinus rhythm and/or to decrease ventricular rhythm. Known drug treatments, however, may not be sufficiently effective, and additional measures must often be taken to control the arrhythmia. Additional measures include ablating cardiac tissue.

One known ablation procedure is generally referred to as the MAZE III procedure and involves electrophysiological mapping of the atria to identify macroreentrant circuits, and then breaking up the identified circuits. These circuits are believed to drive atrial fibrillation, which is disrupted by surgically cutting or burning a maze pattern in the atrium to prevent conduction through these areas. This procedure has been shown to be effective, but it generally requires the use of cardiopulmonary bypass and is a highly invasive procedure associated with high morbidity.

Other procedures have been developed to perform transmural ablation of the heart wall or adjacent tissue walls. Transmural ablation may be grouped into two main categories of procedures: endocardial ablation and epicardial ablation. Endocardial procedures are performed from inside the wall (typically the myocardium) that is to be ablated. Endocardial ablation is generally carried out by delivering one or more ablation devices into the chambers of the heart by catheter delivery, typically through the arteries and/or veins of the patient. In contrast, epicardial procedures are performed from the outside wall (typically the myocardium) of the tissue that is to be ablated. These types of procedures are often performed using devices that are introduced through the chest and between the pericardium and the tissue to be ablated. Epicardial ablation techniques provide the distinct advantage that they may be performed on the beating heart without the use of cardiopulmonary bypass.

When performing procedures to treat atrial fibrillation, an important aspect of an epicardial procedure generally is to isolate the pulmonary veins from the surrounding myocardium. The pulmonary veins connect the lungs to the left atrium of the heart, and join the left atrial wall on the posterior side of the heart. When performing open chest surgery, such as facilitated by a full sternotomy, for example, epicardial ablation may be readily performed to create the requisite lesions for isolation of the pulmonary veins from the surrounding myocardium. Such procedures, however, have been limited by their complexity and morbidity. The location of the pulmonary veins creates significant difficulties during less invasive procedures, since one or more lesions are typically required to be formed to completely encircle the pulmonary veins.

More particularly, FIG. 1 illustrates a heart 10 and designated “left” and “right” relative to the left and right sides of a human patient. FIG. 1 illustrates four pulmonary veins (PV) 12, i.e., a right superior pulmonary vein 12a, a left superior pulmonary vein 12b, a left inferior pulmonary vein 12c, and a right inferior pulmonary vein 12d. A first pericardial reflection 14a extends between the superior vena cava (SVC) 16 and the right superior pulmonary vein 12b, and a second pericardial reflection 14b extends between the right inferior pulmonary vein 12d and the inferior vena cava (IVC) 18.

To ablate tissue around the pulmonary veins 12, an endoscope and endoscopic instruments can be inserted through one or more trocar ports formed in a side of a chest to provide access to the heart 10. During a procedure, the pericardium or the sac surrounding the heart 10 is dissected until the superior vena cava 16 can be visualized through the endoscope. For example, dissection may be performed by carefully scraping a tip or protrusion against the pericardial tissue 20 to separate it with a side-to-side or up-and-down motion of tip.

In known procedures, the pericardial reflection 14a is dissected posterior to the superior vena cava 16, thereby providing an entrance to the transverse pericardial sinus 22. Upon accessing the transverse pericardial sinus 22, other surgical components, such as a snare catheter and retrieval device, can be introduced or wrapped around the pulmonary veins 12, and an ablation device can be drawn to surround the pulmonary veins 12. For this purpose, the second pericardial reflection 14b extending between the right inferior pulmonary vein 12d and the inferior vena cava 18 is also dissected.

More particularly, in one known procedure, an ablation probe is inserted into the space where the pericardial reflection 14a was dissected, wound through the transverse sinus 22 of the pericardium, moved inferiorly along the left side of the heart 10, lateral to the left pulmonary veins 12b and 12c, and along the oblique pericardial sinus 24, thereby completing the path of the ablation probe around three sides of the pulmonary veins 12. The three sides are generally defined between the right superior pulmonary vein 12a and the left superior pulmonary vein 12b, between the left superior pulmonary vein 12b and the left inferior pulmonary vein 12c, and between the left inferior pulmonary vein 12c and the right inferior pulmonary vein 12d. Once positioned, the ablation device is activated to form a lesion around these three sides.

Positioning an ablation probe by dissecting pericardial reflections has been performed effectively in the past (e.g., as described in U.S. Application Publication No. 2004/0111101 and U.S. Application Publication No. 2004/0216748, the contents of which are incorporated herein by reference). However, complications can arise when performing such procedures. More particularly, cutting through the reflection 14a extending between the superior vena cava 16 and the right superior pulmonary vein 12a, presents difficulties and potential complications since the pericardial reflection 14a forms the end of the transverse pericardial sinus 22, and there is no direct way for the endoscopic subxiphoid cannula to approach this pericardial reflection 14a. As a result, it is difficult to visualize the pericardial reflection 14a which, in turn, presents hazards when dissecting the pericardial reflection 14a since the superior vena cava 16, right superior pulmonary vein 12a, and right main pulmonary artery 26 adjacent the aorta 28 are all in the vicinity of this pericardial reflection 14a. Dissection at these sites causes significant concern for surgeons because of the potential injury to the vena cava and resulting hemorrhage.

### SUMMARY

According to one embodiment, an endoscope includes an elongate shaft having a proximal end and a distal end, a lens element attached to the distal end of the elongate shaft and a coupling element. The coupling element extends from the lens element and is configured to hold an operative element such as an ablation element.

According to another embodiment, an endoscope includes an elongate shaft having a proximal end and a distal end, a lens element attached to the distal end of the elongate shaft and a coupling element. The coupling element extends from the lens element and is configured to hold a flexible ablation element in a line of sight of the lens element.

In a further embodiment, a component for use with an endoscope includes a lens element and a coupling element. The lens element is configured to be attached to a distal end of an elongate shaft of the endoscope. The coupling element extends from the lens element and is configured to hold or receive an operative element such as an ablation element.

Another embodiment is directed to a method of forming a lesion in a wall of a heart by epicardial ablation. The method includes inserting a flexible ablation element through an incision formed in the pericardium that surrounds the heart. The method further includes wrapping the flexible ablation element partially around a plurality of pulmonary veins on an epicardial surface of the heart and performing a first ablation of cardiac tissue on the epicardial surface of the heart adjacent to the flexible ablation element. The flexible ablation element is then removed, and the same or a different flexible ablation element is coupled to or supported by a coupling element extending from a lens element attached to a distal end of a shaft of an endoscope. The endoscope having the flexible ablation element coupled thereto is inserted into the body and through the incision, and a second ablation is performed on an epicardial surface of the heart adjacent to a portion of the flexible ablation element coupled to the lens element of the endoscope.

A further embodiment is directed to a method of forming a lesion in a heart of a patient. The method includes positioning an ablation element partially around pulmonary veins on an epicardial surface of the heart and ablating a first section of epicardial tissue adjacent to the ablation element using the ablation element. The method further includes removing the ablation element from the patient, inserting an endoscope supporting the same or a different ablation element into the patient, and ablating a second section of epicardial tissue adjacent to the ablation element.

According to another embodiment, a system for forming a lesion in a heart of a patient includes an endoscopic surgical instrument and an ablation element. The endoscopic surgical instrument may include an elongate shaft having a proximal end and a distal end, a lens element attached to the distal end of the elongate shaft, and a coupling element extending from the lens element. The coupling element is configured to support the ablation element, such as a flexible microwave ablation element.

With embodiments, epicardial ablation may be performed without having to sever or penetrate a pericardial reflection. Embodiments can be performed using multiple ablation stages. During a first ablation or first series of ablations, cardiac tissue around three of four sides of pulmonary veins can be performed, i.e., on a first side superior to a right superior pulmonary vein and a left superior pulmonary vein in a transverse pericardial sinus, on a second side lateral to a left superior pulmonary vein and a left inferior pulmonary vein and on a third side inferior to a left inferior pulmonary vein and a right inferior pulmonary vein in an oblique pericardial sinus. A second ablation can then be performed with an endoscope having a coupling element that holds or supports the same or different ablation element used during the first ablation. During the second ablation, the remaining fourth side of four pulmonary veins is ablated, i.e., lateral to a right superior pulmonary vein and a right inferior pulmonary vein. Thus, more complete ablation patterns can be formed around the pulmonary veins, and these advantages are achieved without having to dissect pericardial reflections.

Embodiments can also be used to ablate other tissue sites. For example, a second ablation or second series of ablations using endoscope embodiments can be performed by placing the ablation element held by the coupling element against a pericardial reflection extending between a right superior pulmonary vein and a superior vena cava and directing energy from the ablation element and to cardiac tissue through the pericardial reflection.

In one or more embodiments, the elongate shaft being substantially rigid, e.g., to withstand forces applied by a surgeon during application of force or positioning the instrument. According to one embodiment, the coupling element is an open-faced coupling element, e.g., a C-shaped coupling element that partially surrounds and supports an ablation element. The coupling element can also be a closed coupling element or loop in which an ablation element can be inserted.

In one or more embodiments, a proximal end of the lens element defines an aperture that is configured to receive the distal end of the elongate shaft. The distal end of the shaft and the lens element having the coupling element can be threadedly secured together. The coupling element can be a separate component that is attached to the lens element. Alternatively, the coupling element being a part of the lens element, e.g., formed or molded as a single piece. Further, the lens element and the coupling element can be made of the same or different materials.

In one or more embodiments, a distal end of the lens element protrudes through a portion of the coupling element. In this manner, the operative element, e.g., ablation element, held, secured or supported by the coupling element and/or tissue to be treated is within the line of sight of a surgeon. Thus, when the surgeon looks into an eyepiece of the endoscope, the surgeon can view the ablation element and/or tissue during treatment.

### BRIEF DESCRIPTION OF THE DRAWINGS

Referring now to the drawings in which like reference numbers represent corresponding parts throughout and in which:

FIG. 1 illustrates a cutaway anterior view of a human heart;

FIG. 2 illustrates an endoscope instrument having a coupling element extending from or attached to a lens element according to one embodiment;

FIG. 3 illustrating mating components of a shaft of an endoscope and a lens element according to on embodiment;

FIG. 4 illustrates an endoscope instrument having an open coupling element extending from or attached to a lens element according to one embodiment;

FIG. 5 is an expanded view of the lens element and open coupling element as shown in FIG. 5;

FIG. 6 is a front view of an open coupling element according to one embodiment;

FIG. 7 illustrates one microwave ablation probe that can be used with embodiments;

FIG. 8 illustrates a coupling element extending from a lens element of an endoscopic instrument holding, receiving or supporting an ablation element according to one embodiment;

FIG. 9 illustrates a method of ablating tissue using an ablation probe held, received or supported by an open coupling element extending from a lens element of an endoscopic instrument;

FIG. 10 is an expanded view of a lens element and a closed coupling element according to another embodiment;

FIG. 11 is a front view of a closed coupling element according to one embodiment;

FIG. 12 illustrates a method of ablating tissue using an ablation probe held, received or supported by a closed coupling element extending from a lens element of an endoscopic instrument;

FIG. 13 illustrates an ablation probe held, received or supported by a closed coupling element extending from a lens element of an endoscopic instrument and being secured to the coupling element by sutures;

FIG. 14 illustrates an example of a surgical instrument that can be used in conjunction with embodiments;

FIG. 15 illustrates an example of a snare catheter or guide that can be used in conjunction with embodiments;

FIG. 16 illustrates a ball-tipped retrieval tool that can be used in conjunction with embodiments;

FIG. 17A illustrates a cutaway anterior view of a human heart and a snare catheter having been advanced around pulmonary veins without dissection of a pericardial reflection;

FIG. 17B further illustrates a human heart and a snare catheter having been advanced around pulmonary veins without dissection of a pericardial reflection;

FIG. 18 illustrates lesions formed on three sides around pulmonary veins;

FIG. 19 illustrations application of an ablation element held, receive or supported by coupling element extending from a lens element of an endoscopic instrument to perform ablation around a fourth side between superior and inferior right pulmonary veins according to one embodiment;

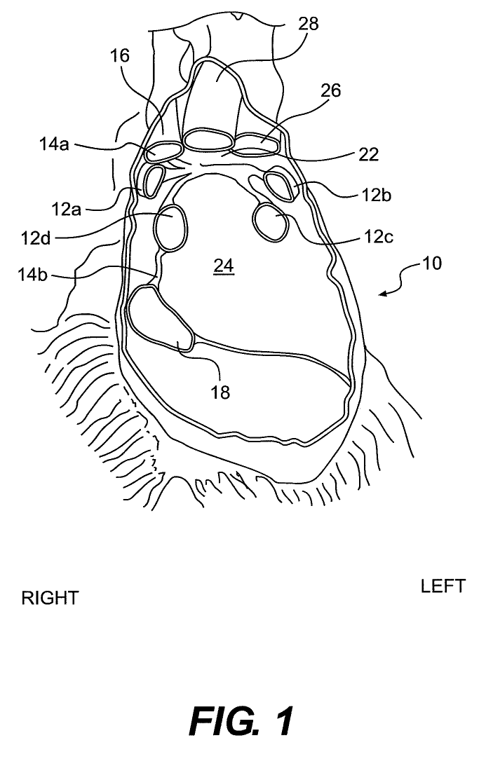

### DETAILED DESCRIPTION OF ILLUSTRATED EMBODIMENTS

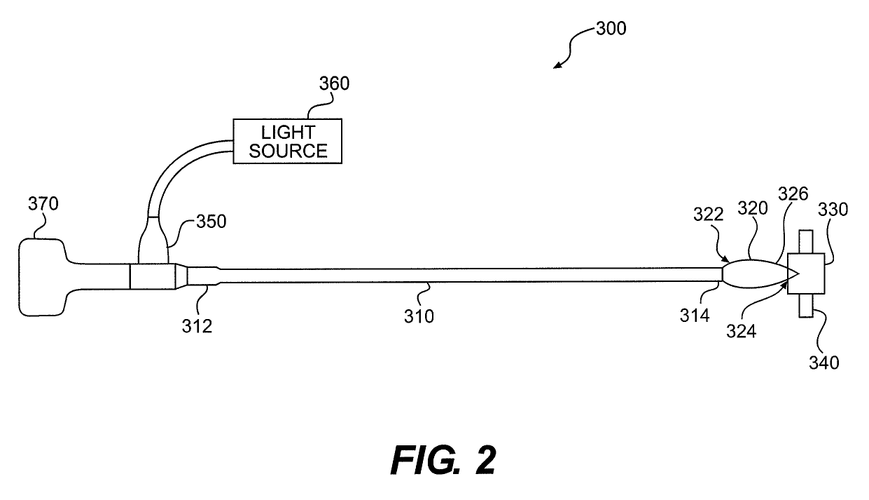

Referring to FIG. 2, according to one embodiment, an endoscope 300 or other support device, instrument or handle capable of visualization includes an elongate shaft 310 having a proximal end 312 and a distal end 314, a lens element 320 having a proximal end 322 and a distal end 324 and a coupling element, attachment element or crosspiece 330 (generally referred to as a coupling element 330). The coupling element 330 is attached to or extends from the lens element 320.

The lens element 320 can be a visualization lens, e.g., a FLEXview visualization lens, a FLEXview Unilateral routing lens, or a lens element/dissection tip or cone, all of which are available from Boston Scientific Corporation, or another suitable lens element. Further aspects of suitable lens elements 320 and the general types of lens elements and endoscope instruments that can be used or adapted for use with embodiments are provided in “Guidant FLEXview™ Visualization Lens, EL2049072 (Sep. 16, 2005), and “Guidant FLEXview® Unilateral Routing System, EL2050411 Rev. B (Feb. 27, 2006), VasoView® 7 xB™ Endoscopic Vessel Harvesting System, EL 2047992 Rev. C (Oct. 25, 2005), and VasoView® 7 xS™ Endoscopic Vessel Harvesting System, EL 2047993 Rev. B (Sep. 30, 2005), the contents of all of which are incorporated herein by reference. This specification generally refers to a lens element 320 for purposes of explanation, and it should be understood that lens elements 320 of various configurations can be utilized.

The endoscope 300 also includes an illumination port 350 that is attachable to a light source 360 and an eyepiece 370 through which a surgeon can view anatomical structures, tissue and/or the operative element 340 adjacent to the lens element 320. The endoscope 300 allows a surgeon to place an operative element 340, such as an ablation element, that is held or secured by or received within the coupling element 330, against target tissue and to simultaneously visualize the target tissue and/or operative element 340 during ablation of the tissue.

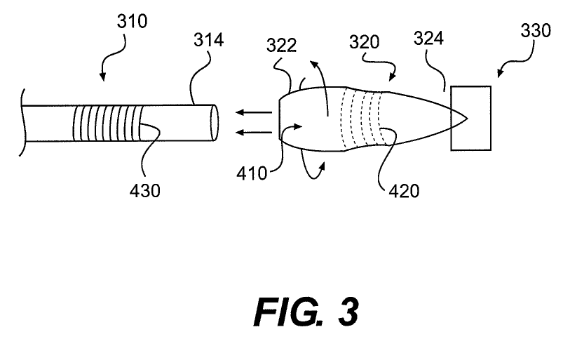

With further reference to FIG. 3, the lens element 320 is attached to and extends from the distal end 314 of the elongate shaft 310. The lens element 320 and the coupling element 330 extending there from can be attached to the distal end 314 of the shaft 310 in various ways. For example, as shown in FIG. 3, one suitable lens element 320 defines an aperture 410 and has a threaded internal surface 420. The distal end 314 of the shaft 310 has a threaded outer surface 430 so that the distal end 314 of the shaft 310 can be inserted into the aperture 410 defined by the lens element 320, and the lens element 320 and the coupling element 330 attached thereto can be threadedly secured to the distal end 314 of the shaft 310. The lens element 320 and coupling element 330 can also be attached to the shaft 310 in other ways, e.g. using snap fit components. Thus, FIG. 3 is provided as one example to illustrate how a shaft 310 of the endoscope 300 and the lens element 320 can be coupled together.

Referring again to FIG. 2, the coupling element 330 extends from or is attached to the lens element 320. The coupling element 330 is configured to hold or secure an operative element or device 340 (generally illustrated in FIG. 2) at the distal end 324 of the lens element 320. The endoscope 300 is manipulated by a surgeon so that the operative element 340 is positioned against the tissue to be treated. For this purpose, the shaft 310 can be stainless steel or other suitable materials and be substantially rigid or malleable so that the shaft 310 has sufficient strength to withstand forces resulting from positioning and manipulating the endoscope 300.

For example, the shaft 310 should have sufficient compressive strength to permit a surgeon to push against the endoscope 300 to force the coupling element 330 and/or operative element 340 held thereby against target tissue. More particularly, during use, a surgeon inserts the endoscope 300 into a patient through an incision or trocar, positions the operative element 340 held by the coupling element 330 adjacent to the tissue to be treated, applies the necessary amount of pressure to push the operative element 340 against the target tissue or to position the operative element 340 sufficiently close to the target tissue, and treats target tissue adjacent to the operative element 340. The surgeon can view the tissue and/or the operative element 340 through the eyepiece 370 and lens element 320 during treatment.

According to one embodiment, the coupling element 330 is used to hold or secure an operative element 340 that is a tissue ablation element, which is attached to a suitable energy source (not shown in FIG. 2). Embodiments can be used with various ablation elements 340 including, for example, microwave, laser, radio frequency and high intensity focused ultrasound ablation elements.

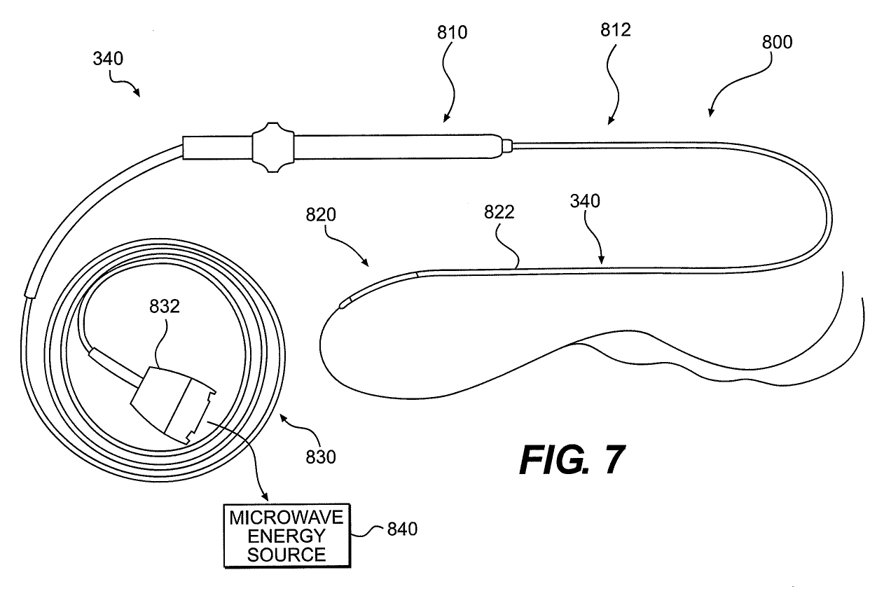

According to one embodiment, the coupling element 330 is configured to hold or secure an operative element 340 in the form of a microwave ablation element or probe. One example of a suitable microwave ablation element or probe is the FLEX 10® XE ablation probe available from Boston Scientific Corporation (discussed in further detail below with reference to FIG. 7). Further aspects of the FLEX 10® XE ablation probe are provided in “Guidant FLEX 10® XE Ablation Probe for the Microwave Ablation System,” EL2052892 (Oct. 26, 2006). For ease of explanation and illustration, reference is made to a flexible microwave ablation element 340 as the operative element that is held or secured to the coupling element 330; however, embodiments can be used with other microwave ablation probes other than the FLEX 10® XE ablation probe, other types of ablation probes other than microwave ablation probe, and with other operative elements other than ablation elements, for use in other applications to treat tissue other than cardiac tissue.

The coupling element 330 attached to or extending from the lens element 320 can have various shapes and sizes, and the coupling element 330 can be configured to hold or secure different ablation elements 340. In the embodiment illustrated in FIGS. 2 and 3, the lens element 320 has a generally conical shape with a sloped or conical distal surface 326. In the illustrated embodiment, the coupling element 330 extends from or is attached to the surface 326. Other lens element 320—coupling element 330 configurations can also be utilized depending on, for example, the shape and size of the lens element 320 and the shape and size of the coupling element 330.

Depending on the particular configuration, the lens element 320 and the coupling element 330 can be made of the same or different materials. In one embodiment, the lens element 320 and the coupling element 330 are the same material. In another embodiment, the coupling element 330 can be a separate component that is attached or fixed to the lens element 320, and the lens element 320 and the coupling element 330 may or may not be the same material. In a further embodiment, the lens and coupling elements 320 and 330 can be an integrated component, e.g., molded or formed as a single component and the lens and coupling elements 320 and 330 may be the same material.

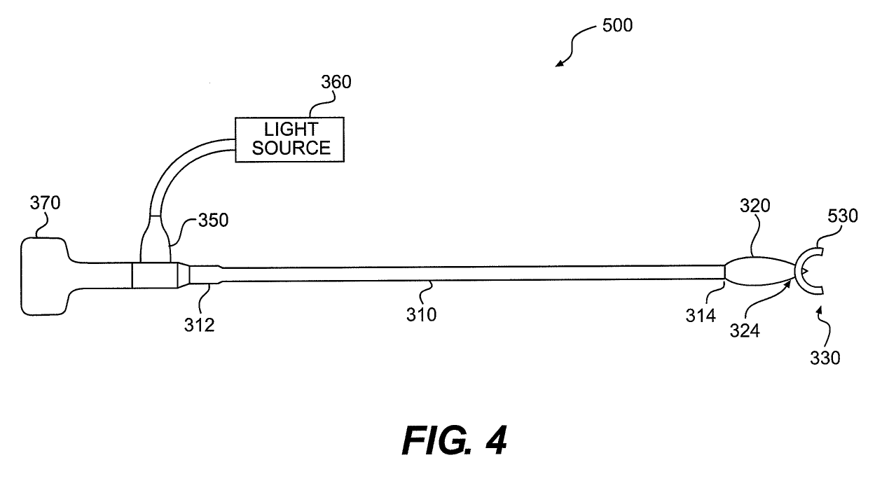

Referring to FIG. 4, according to one embodiment, an endoscope 500 or other instrument, handle or device capable of visualization includes a coupling element 330 in the form of an open coupling member or channel 530, e.g. a C-shaped channel (generally referred to as a “channel”). The channel 530 is configured to partially surround, receive or hold an ablation element 340. For example, a C-shaped channel may be particularly suitable for receiving or holding a flexible ablation element of the Flex 10® XE ablation probe, which also has a generally C-like shape that can be received by a C-shaped coupling element 330.

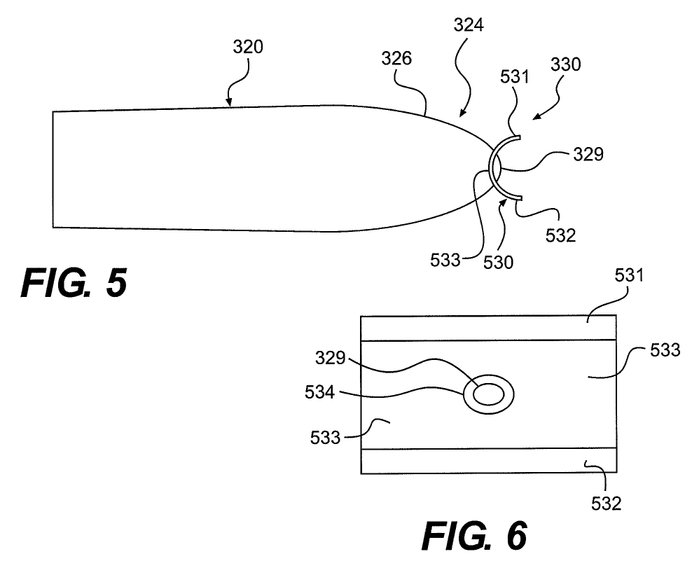

With further reference to FIGS. 5 and 6, the channel 530 includes an upper channel member or portion 531, a lower channel member or portion 532 and an intermediate channel portion 533 extending there between. In the illustrated embodiment, the distal end or tip 329 of the lens element 320 protrudes through an aperture 534 formed through the coupling element 530, e.g., through the intermediate channel section 533. An aperture 534 can be formed in other portions of a coupling element 530 depending on, for example, the shape and size of the coupling element 530 and the configuration of the distal tip 329 of the lens element 320.

These configurations advantageously allow visualization by a surgeon through the lens element 320 extending through the channel 530 to view target tissue and/or an operative element 340 held by the channel 530. More particularly, the ablation element 340 can be held by the channel 530 so that the ablation element 340 is within the line of sight of the surgeon when the surgeon looks through the eyepiece 370 and the lens element 320. In this manner, a surgeon can advantageously view an ablation element 340 and/or tissue adjacent the channel 530, thereby allowing a surgeon to simultaneously treat tissue while viewing the treated tissue and/or the ablation element 340 held or received by the channel 530 at the distal end 324 of the lens element 320. For example, a surgeon can determine how treatment is progressing based on a change of color of the tissue during ablation. In alternative embodiments, the distal tip 329 of the lens element 320 does not extend through the coupling element 320. In such cases, visualization through the lens element 320 and the coupling element 330 can be accomplished by selection of appropriate lens element 320 and channel 530 materials having suitable transparent properties.

Referring to FIGS. 7-9, during use, an ablation element or probe 340 held by the coupling element 330 can be positioned adjacent to or against cardiac tissue to be ablated. As discussed above, one example of a suitable ablation probe 340 that can be utilized with embodiments is a flexible microwave ablation probe 800 such as the FLEX 10® XE ablation probe 800 (generally illustrated in FIG. 7), available from Boston Scientific Corporation. The illustrated microwave ablation probe 800 includes a handle 810, a shaft 812 extending from the handle 810, and a flexible sheath 820 extending from the shaft 810. The flexible sheath 820 includes one or more microwave ablation elements 822 (identified by black markers), which can be selectively activated to ablate tissue. A cable 830 extends from a proximal end of the handle 810 and terminates at a connector 832 for connection to a source 840 of microwave energy, which drives selected microwave ablation elements 822.

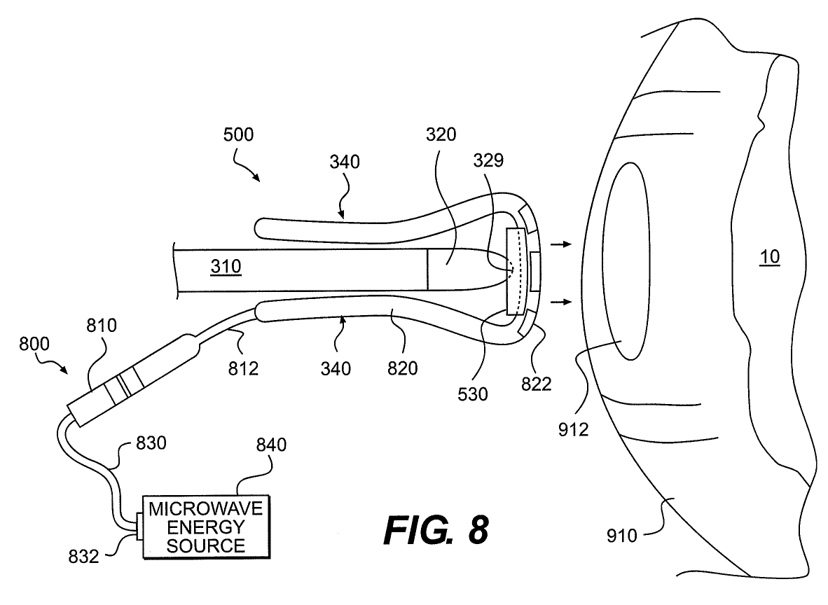

Referring to FIG. 8, during use, the flexible sheath 820 having one or more ablation elements or sections 822 is wrapped around the distal end of the endoscope 500. More particularly, the sheath 820 is received by or partially surrounded by the coupling element 330. In the illustrated embodiment, the sheath 820 is held or received by a C-shaped channel 530 (as shown in FIG. 5) so that a portion of the sheath 820 is surrounded or received by the channel 530, and another portion of the sheath 820 is exposed. Other coupling element 330 configurations can be utilized to adapt to different ablation element configurations and ablation applications.

The assembly of the endoscope 500 and the sheath 820 that is wrapped around and received or held by the channel 530 is inserted into a patient 910 through an incision or trocar (generally illustrated as 912). If necessary, the size of the incision or trocar 912 can be increased to accommodate the width of the endoscope 500 having the sheath 820 wrapped around the distal end thereof since the width of the instrument inserted through the trocar 912 is larger than the shaft 310 due to the coupling element or channel 530 and the ablation element 340 extending beyond the edges or sides of the channel 530.

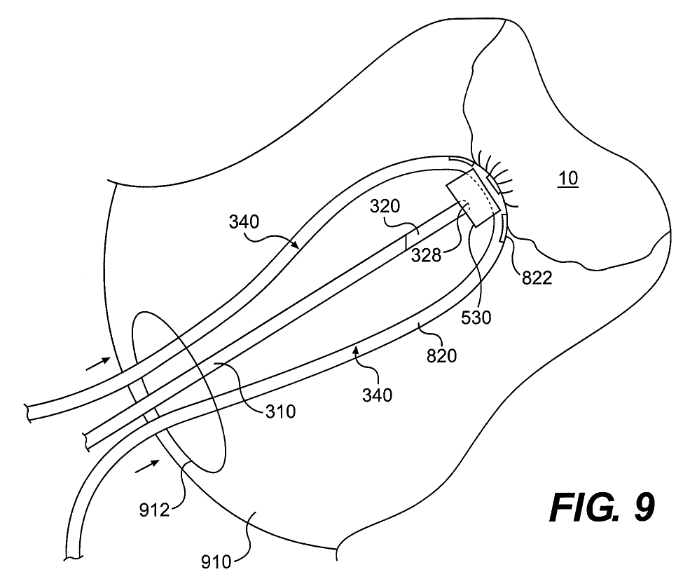

Referring to FIG. 9, the endoscope 500 and sheath 820 assembly inserted through the trocar 912 is advanced into the body and maneuvered by the surgeon so that one or more ablation elements 822 at the distal end of the endoscope 500 contact, or are sufficiently close to, target cardiac tissue 10. For this purpose, the surgeon can apply sufficient force to position the distal end of the shaft 310 holding the sheath 820 against the cardiac tissue 10. After the one or more ablation elements 822 are placed in a desired position, microwave energy from the energy source 840 can be applied to one or more of the elements 822 to ablate selected epicardial surfaces of the heart adjacent to the ablation elements 822. In the embodiments illustrated in FIGS. 8 and 9, the coupling element 320 is in the form of an open channel 530, which holds the sheath 820 to allow direct contact between one or more ablation elements 822 and target cardiac tissue 10.

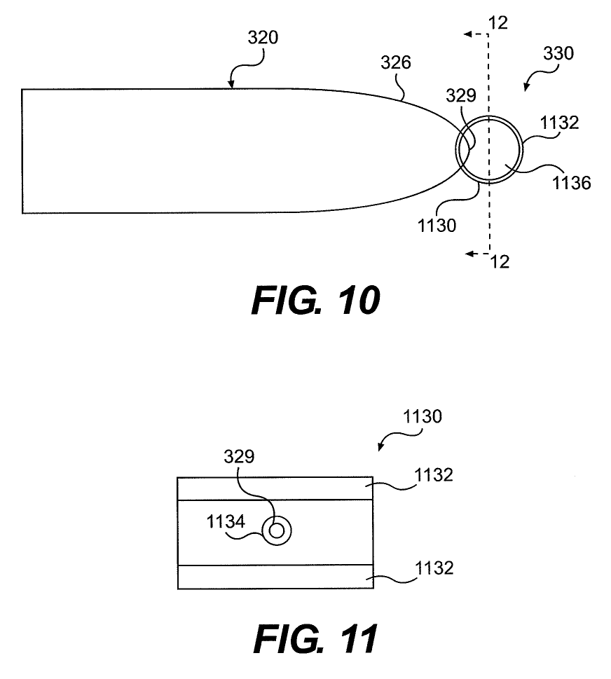

Referring to FIGS. 10 and 11 according to another embodiment, the coupling element 330 is in the form of a closed coupling element 1130, e.g., a closed loop or ring. The closed coupling element 1130 has an outer body 1132 that defines a lumen or channel 1136. An ablation element 340, such as the flexible sheath 822 of the microwave ablation probe 800 shown in FIG. 7, can be inserted through the lumen 1136 and wrapped around the distal end of the instrument 300.

In the illustrated embodiment, the distal tip 329 of the lens element 320 protrudes through an aperture 1134 defined by the coupling element 1130. Thus, the flexible sheath 822 held within the lumen or channel 1136 is within the line of sight of the surgeon when the surgeon looks through the eyepiece 370 and the lens element 320. In this manner, a surgeon can view the ablation elements 822 adjacent to the closed coupling element 1130, thereby allowing a surgeon to simultaneously treat tissue while viewing the ablation element 800 held or received by the closed coupling element 1130 at the distal end 324 of the lens element 320. In alternative embodiments, the distal tip 329 of the lens element 320 does not extend through the body 1132 of the coupling element 1130, and visualization through the lens element 320 and the body 1132 can be accomplished by selection of lens element 320 and body 1132 materials have sufficient transparency.

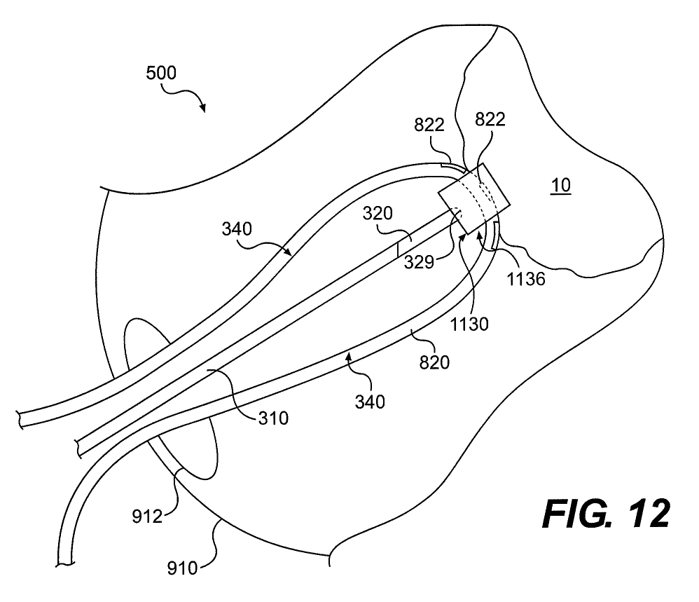

Referring to FIG. 12, during use, the assembly of the endoscope 500 and the sheath 820 inserted through the lumen 1136 can then be is inserted into a patient 910 through an incision or trocar 912, as discussed above with reference to FIGS. 7-9, so that one or more ablation elements 822 contact or are sufficiently close to tissue to be ablated. One or more microwave ablation elements 822 can then be activated to ablate selected epicardial surfaces of the heart 10 adjacent to the ablation element 10 held within the closed coupling element 1130.

In the illustrated embodiment, the coupling element 1130 completely surrounds the sheath 820 carrying the ablation elements 822. Thus, ablation elements 822 positioned outside of the coupling element 1130 may directly contact cardiac tissue, while ablation elements 822 inside the coupling element 1130 may not. The coupling element 1130 can be made of a suitable material that conducts sufficient heat so that heat generated by an ablation element 822 positioned inside the coupling element 1130. Additionally, or alternatively, ablation can be performed by other ablation elements 822 that are located outside of the coupling element 1130 and that may or may not directly contact the tissue. Thus, ablation can be performed by ablation elements 822 positioned inside and/or outside of the coupling element 1130 as needed. The particular manner in which tissue is ablated may vary depending on, for example, the design, shape and/or materials of the coupling element 1130 and the number and spacing of ablation elements 822 on the sheath 820. For example, although the Figures illustrate a coupling element 340 as having a circular or an arcuate shape, the coupling element 340 can have other shapes, such square or rectangular shapes or linear portions. Further,

Further, although FIGS. 4-6 and 8-12 illustrate coupling elements 330 in the form of an open C-channel 530 that partially surrounds a sheath 820 or an ablation element 822 and a closed ring or loop 1130 that completely surrounds a sheath 820 or an ablation element 822, embodiments can be used with other coupling elements 330 having different configurations. For example, in other embodiments, the coupling element 330 can be a clamp-like or clip-like structure such as a compression spring clamp member that is biased to a closed position and requires application of force to open the clamp member. With this embodiment, a surgeon can hold the clamp member open, and the ablation element 340 can be inserted between clamps. The clamp member can then be released to clamp onto a segment of the ablation element 340. The endoscope 300 having the ablation element 340 clamped at a distal end thereof can then be inserted into a patient 910 through a trocar 912 or incision in order to ablate tissue.

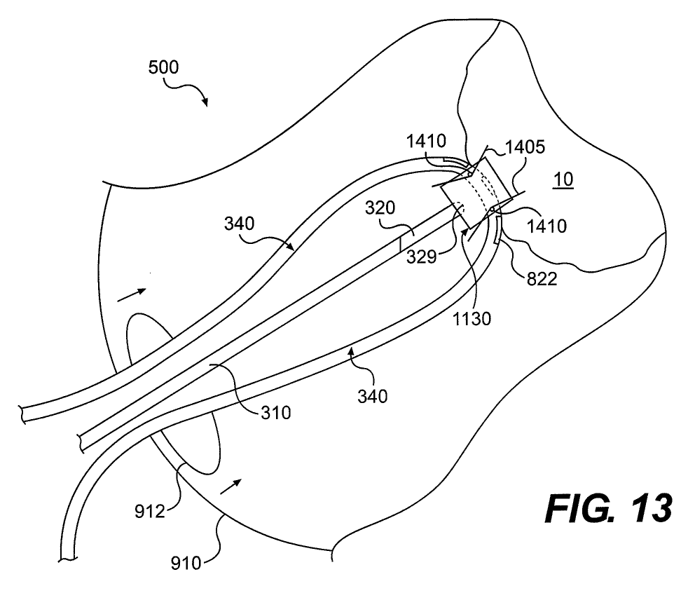

Referring to FIG. 13, if necessary, a coupling element 330, e.g., coupling element 530 or 1130, can include one or more apertures 1410 through which sutures 1405 can be inserted to tie or fasten an ablation element 340 to the coupling element 330. In other embodiments, other fasteners besides sutures 1405 can be utilized, e.g., set screws and locking bands. These additional fasteners can be used to securely attach the ablation element 340 to the coupling element 330.

Embodiments can be used in conjunction with various other devices and surgical methods in order to allow a surgeon to manually position an operative element and treat or ablate selected portions different types of tissue. Following is a description of a method of treating atrial fibrillation by ablating atrial cardiac tissue without having to dissect pericardial reflections 14a and 14b as is done in known methods using known ablation devices. However, it should be appreciated that embodiments can be used in other applications and in conjunction with other surgical devices to treat and/or ablate other types of tissue.

Initially, at least one trocar port or opening 912, e.g., a 12 mm trocar port, is formed in the right chest of the patient (e.g., a port though the third intercostal space of the right chest) to provide access to the heart 10 by a known endoscopic instrument. Embodiments can be used with procedures that involve one or multiple trocar ports. For example, rather than a single trocar port, a procedure can be carried out using two 12 mm trocar ports that are inserted in the right chest in the third and fourth intercostals space.

An operating endoscope or other suitable endoscopic instrument with an endoscopic grasper can be inserted through the endoscope's incorporated exit ports or working channel through a trocar port 912, and a pair of endoscopic shears or scissors (e.g. as shown in VasoView® 7 xS™ Endoscopic Vessel Harvesting System, EL 2047993 Rev. B (Sep. 30, 2005)) can be inserted through another trocar port 912. Carbon dioxide gas insufflation may be conducted in the right pleural cavity to collapse the right lung and allow the surgeon to incise the pericardium that surrounds the heart 10 in longitudinal fashion, anterior to the right phrenic nerve. The operating endoscope and endoscopic shears are removed from the body, as well as one of the 12 mm trocar ports.

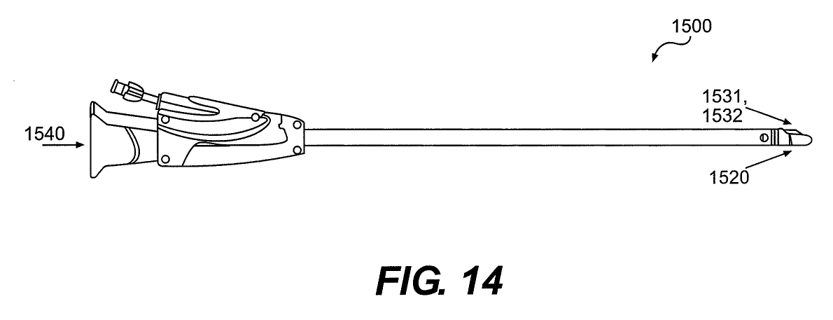

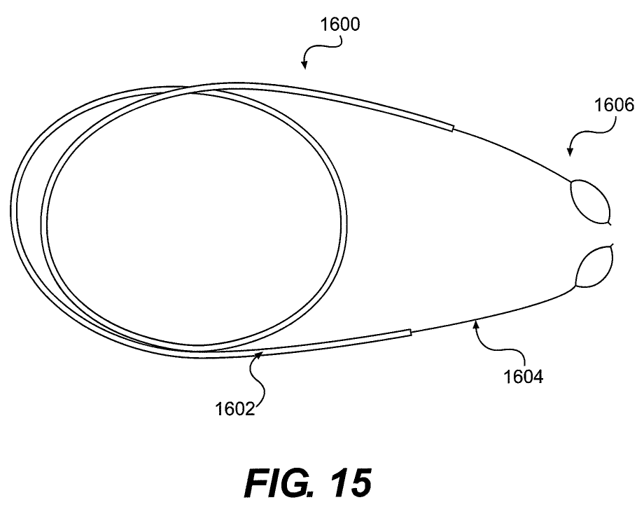

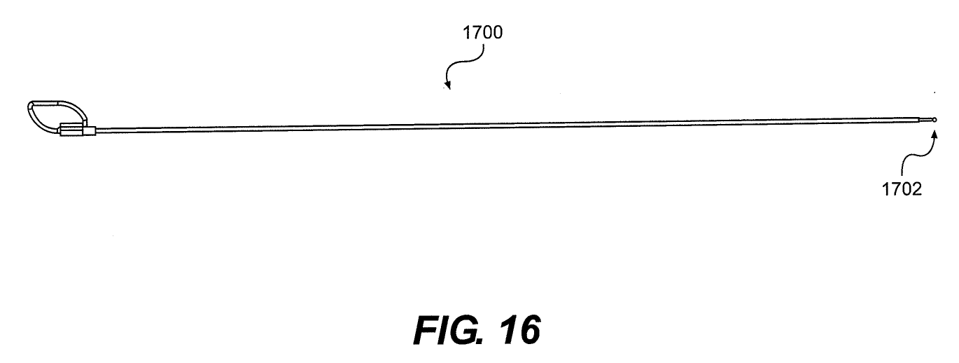

Referring to FIGS. 14-16, an endoscope with a ball tip (e.g., as described in U.S. Publication No. 2006/0270900, the contents of which are incorporated herein by reference) or an instrument having a ball tip accessory or retrieval tool, such as the FLEXview Cannula 1500 (generally illustrated in FIG. 14) available form Boston Scientific Corporation, can be utilized to deliver accessories for inserting and positioning of an ablation element 340. As shown in FIG. 14, for example, the cannula device 1500 includes a visualization lens 1520 and distal exit ports or working channels 1531 and 1532 for delivering accessory tools, such as a snare loop (one example of which is shown in FIG. 15) and retrieval tool 1700 with a ball tip distal end 1702 (one example of which is shown in FIG. 16). An endoscopic instrument, such as a 7 mm extended length endoscope available from Boston Scientific Corporation, is inserted into the bell 1540 of the cannula 1500. Further aspects of an example of a cannula device 1500 that can be used in conjunction with embodiments and the components illustrated in FIGS. 15 and 16 are described in “Guidant FLEXView™ System, EL2053858 (Sep. 5, 2006), the contents of which are incorporated herein by reference. While this specification refers to examples of suitable instruments that can be used in conjunction with or adapted for use with embodiments, it should be understood that various other endoscopic instruments can also be utilized to perform these functions. For ease of explanation, reference is made generally to a ball-tipped endoscope or accessory.

Referring again to FIG. 15, one suitable snare catheter 1600 that can be used in this procedure includes a snare guide 1602 and a snare wire 1604 having loops 1606 at the ends thereof. A snare catheter 1600 may be constructed of flexible plastic material such as polyethylene, polytetratrial fibrillationluoroethylene (PTFE, e.g., TEFLON®), polyvinyl chloride, nylon, or the like. The catheter 1600 is tubular, to allow suture line or wire 1604 to pass therethrough. The wire 1604 includes a suture loop 1606 formed with a sliding knot (an Endoloop) in a distal end thereof. The loop 1606 is located distally of the distal end of catheter 1600. Suture loop 1606 may be formed from a conventional suture material or braided stainless steel wire cable, for example. Alternatively, the entire suture line may be made of NITINOL®, or other nickel-titanium alloy without the need to use a sliding knot. One suitable snare catheter 1600 that can be utilized is the FLEXview Routing Snare, available from Boston Scientific Corporation.

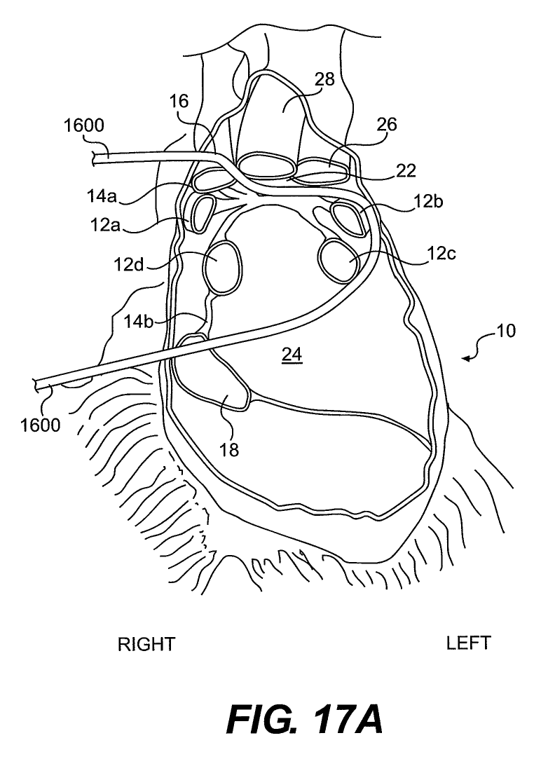

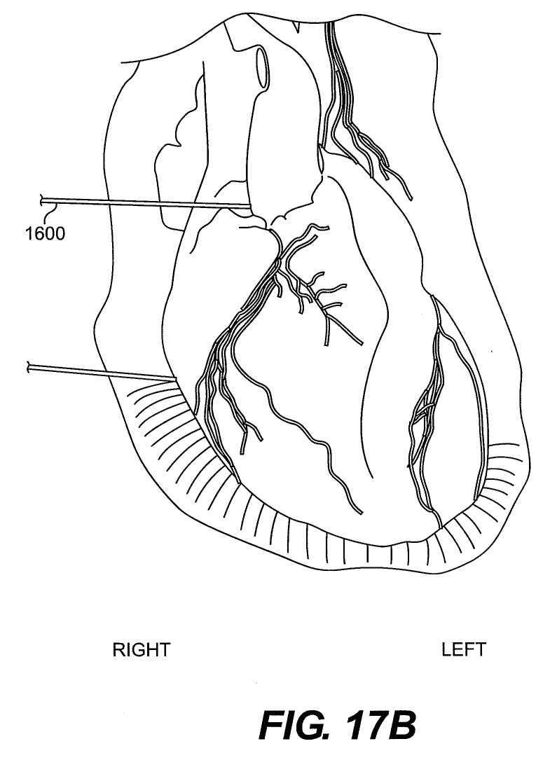

As generally illustrated in FIGS. 17A-B, the ball tip is inserted anterior to the surface of the heart 10, and moved to the region of the right atrial appendage. The ball tipped endoscope is inserted into the groove between the medial border of the right atrial appendage and the right atrium, and the ascending aorta 28 is identified. The ball tip is moved inferiorly along the posterior border of the ascending aorta 28, until the opening of the transverse pericardial sinus 22 is visualized. The ball tipped endoscope is advanced through the transverse pericardial sinus 22, past the pulmonary artery 26 and the base of the left atrial appendage, until the left side of the pericardium is visualized.

The snare catheter 1600 is detached from the ball tipped endoscope, the snare loop 1606 is retracted into the catheter body or guide 1602, and the catheter 1600 is advanced to the left surface of the pericardium. Upon continued advancement, the catheter 1600 deflects inferiorly or downwardly along the left side of the heart 10, lateral to the left superior and left inferior pulmonary veins 12b and 12c, until the catheter 1600 lies in the oblique pericardial sinus 24 on the posterior aspect of the heart 10. The ball tipped endoscope is pulled out of its position in the transverse pericardial sinus 24, advanced anterior to the heart 10, then moved inferiorly along the anterior surface of the heart 10 until the catheter 1600 reaches the apex of the heart. The ball tipped endoscope continues to be moved past the apex to the posterior aspect of the heart 10. Thus, the endoscope now lies in the oblique pericardial sinus 24 where the distal end of the snare catheter 1600 can be visualized.

The wire loop 1606 is extended outwardly from the catheter 1600. A ball tip is used to retrieve or attached to the loop 1606 of the catheter 1600. The ball tip inserted through the loop 1606, and the loop 1606 is tightened to attach the snare catheter 1600 to the ball tipped endoscope. The ball tipped endoscope is then pulled out of the body, and the catheter 1600 is detached from the endoscope. The distal end of an operative element 340, such as a flexible microwave ablation probe (e.g., as shown in FIG. 7) is then attached to the proximal end of the snare catheter 1600. For example, with a microwave ablation probe, such as the FLEX 100 XE ablation probe available from Boston Scientific Corporation, suture ties can be attached to the distal end of the probe may be tied onto the ablation probe into position, coursing through the transverse pericardial sinus 24.

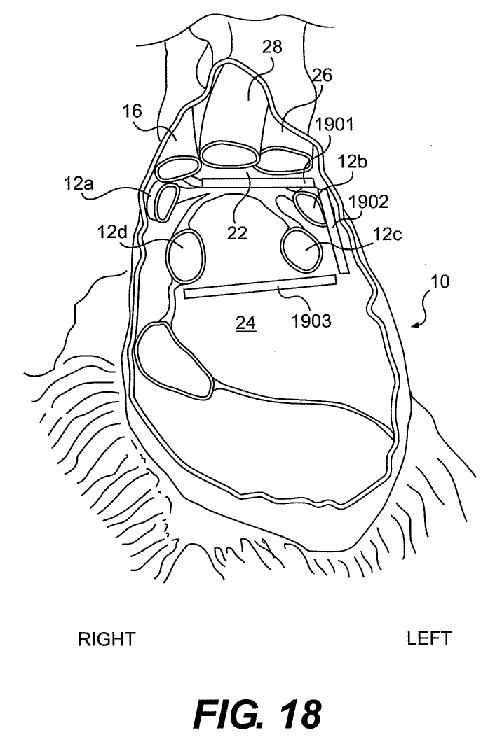

Referring to FIG. 18, ablation elements 822 on the sheath 820 of the ablation probe 800 are activated in order to ablate atrial tissue on three sides of the pulmonary veins 12 (generally illustrated as lesions 1901, 1902 and 1903). More particularly, lesion 1901 is formed in superior tissue between the right and left superior pulmonary veins 12a and 12b, lesions 1902 is formed in tissue lateral of the left superior pulmonary vein 12b and the left inferior pulmonary vein 12c, and lesions 1903 is formed in tissue inferior to the space between the left and right inferior pulmonary veins 12c and 12d and in the oblique pericardial sinus 24. Although FIG. 18 illustrates three separate lesions 1901-1903 for purposes of illustration, a single lesion may also be formed around the pulmonary veins.

Following ablation around three sides of the four pulmonary veins 12, the flexible microwave ablation probe 800 is removed from the body, and the same or a different microwave ablation probe (e.g., flexible microwave ablation probe 800), or another suitable ablation element 340, is attached or inserted into the coupling element or crosspiece 330 extending from the lens element 320 of an endoscope 300, e.g., the coupling elements 530 and 1130 as shown in FIGS. 7, 8, 11 and 12. The distal segment of the instrument 300 having the flexible sheath 820 supported by or attached to the coupling element 330 is then inserted into the body and against tissue to be ablated. If necessary, the incision may be extended slightly to accommodate the width of the device.

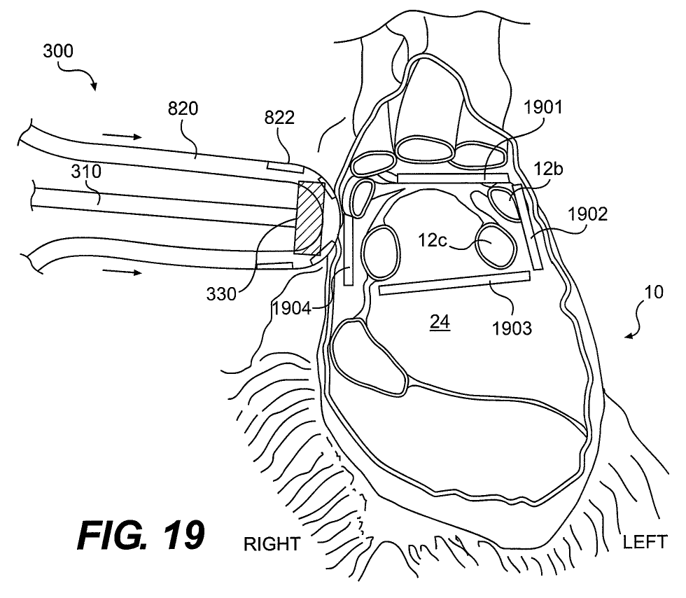

For example, referring to FIG. 19, the sheath 820 of the ablation probe 800 can be inserted through the pericardial incision and placed along the posterior border of the vena cava to ablate the left atrial tissue 1900 lateral to the right superior pulmonary vein 12a and the right inferior pulmonary vein 12d to form a lesion 1904, which was not formed previously when the ablation probe 800 was wrapped around the pulmonary veins 12. In this manner, embodiments allow formation of more complete lesions around pulmonary veins 12.

Other tissue sites can also be ablated with embodiments. For example, the probe 300 may then be used to ablate left atrial tissue immediately medial to the superior vena cava 16. For this purpose, the probe 300 can be placed in a notch between the superior vena cava 16 and the aorta 28, pushed against the tissue surface, and activated to perform ablation. The probe 300 can also be used to ablate left atrial tissue immediately medial to the inferior vena cava 18. For this purpose, the probe 300 is advanced anterior to the inferior vena cava 18, into the oblique pericardial sinus 24, and pulled back slightly until the probe 300 is in contact with the medial border of the inferior vena cava 18. Further, embodiments can be used to ablate another tissue site at which dissection of the pericardial reflections 14a and 14b are is normally performed posterior to the superior vena cava 16 and inferior vena cava 18. For this purpose, the ablation probe 300 is placed against the pericardial reflection at these two points, and pushed medially to direct energy through the thin pericardial layer and into the left atrial tissue beyond the pericardial layer.

Thus, with apparatus and method embodiments, more complete pulmonary vein 12 isolation can be achieved epicardially without the need for surgical dissection through pericardial reflection, thereby advantageously reducing or eliminating risk of injury to the vena cava and resulting hemorrhage that may result from puncture of the vena cava or atrium.

Although the anatomical structures and individual components described herein are known and would be readily understood by those of ordinary skill in the art reading the present disclosure and referring to the Figs. herein, additional views may be found in United States Application Publication No. US2004/0111101 A1 and United States Application Publication No. 2006/0270900, the contents of which are incorporated herein by reference.

Further, although this specification refers to an endoscope having a coupling element configured to hold or secure the Flex 10® microwave probe embodiments of the invention are not limited to use of this product only. Endoscope embodiments having a coupling element or crosspiece can be used with other ablation devices configured to form a long linear lesion and which are sufficiently flexible to surround the pulmonary veins as described herein may be substituted. Further, endoscope embodiments having a coupling element or crosspiece can be used with other ablation devices that are designed or used for ablation of tissue besides cardiac tissue. Further, the energy type for performing the ablation need not be microwave energy, but may alternatively be any of the other types of energy that have been used to form lesions (e.g., RF, electrical, heat, chemical, ultrasonic, etc.).

What is claimed is:

1. An endoscopic surgical instrument, comprising:
an elongate shaft having a proximal end and a distal end;
a lens element attached to the distal end of the elongate shaft; and
a coupling element extending from the lens element, the coupling element being configured to support an ablation element.

2. The endoscopic surgical instrument of claim 1, wherein the elongate shaft is substantially rigid.

3. The endoscopic surgical instrument of claim 1, wherein a proximal end of the lens element defines an aperture configured to receive the distal end of the elongate shaft.

4. The endoscopic surgical instrument of claim 1, wherein the lens element has a threaded inner surface, the distal end of the elongate shaft having a threaded outer surface, and the lens element being configured to be threadedly secured to the distal end of the elongate shaft.

5. The endoscopic surgical instrument of claim 1, wherein the coupling element is configured to receive the ablation element.

6. The endoscopic surgical instrument of claim 1, wherein the coupling element is an open-faced coupling element.

7. The endoscopic surgical instrument of claim 1, wherein the coupling element is a closed coupling element, and the ablation element is insertable through an aperture defined by the closed coupling element.

8. The endoscopic surgical instrument of claim 1, wherein the coupling element is attached to the lens element.

9. The endoscopic surgical instrument of claim 1, wherein the coupling element is a part of the lens element.

10. The endoscopic surgical instrument of claim 1, wherein the lens element and the coupling element are made of the same material.

11. The endoscopic surgical instrument of claim 1, wherein a distal end of the lens element protrudes through a portion of the coupling element.

12. The endoscopic surgical instrument of claim 1, wherein the coupling element is configured to hold a flexible microwave ablation element.

13. The endoscopic surgical instrument of claim 1, wherein a width of the coupling element is greater than a width of the lens element.

14. An endoscopic surgical instrument, comprising:
an elongate shaft having a proximal end and a distal end;
a lens element attached to the distal end of the elongate shaft; and
a coupling element extending from the lens element and being configured to hold a flexible ablation element in a line of sight of the lens element.

15. The endoscopic surgical instrument of claim 14, wherein a distal end of the lens element protrudes through the coupling element.

16. An apparatus for use with an endoscopic surgical instrument, comprising:
a lens element configured for attachment to a distal end of an elongate shaft of the endoscope; and
a coupling element extending from the lens element, the coupling element being configured to hold or receive an ablation element.

17. A method of forming a lesion in a wall of a heart of a patient by epicardial ablation, the heart being surrounded by a pericardium, the method comprising:
inserting a flexible ablation element through an incision formed in the pericardium surrounding the heart;
wrapping the flexible ablation element partially around a plurality of pulmonary veins on an epicardial surface of the heart;
performing a first ablation of cardiac tissue on the epicardial surface of the heart adjacent to the flexible ablation element;
removing the flexible ablation element;
coupling the same or a different flexible ablation element to a distal end of a lens element of an elongate shaft of an endoscope;
inserting the endoscope having the flexible ablation element coupled thereto into the patient through the incision; and
performing a second ablation of cardiac tissue on an epicardial surface adjacent to a portion of the flexible ablation element coupled to the lens element of the endoscope.

18. The method of claim 17, wherein a pericardial reflection is not severed or penetrated.

19. The method of claim 17, wherein performing the first ablation includes ablating cardiac tissue on three of four sides of four pulmonary veins.

20. The method of claim 19, wherein performing the second ablation includes ablating cardiac tissue on the remaining fourth side of four pulmonary veins.

21. The method of claim 19, wherein performing the first ablation comprises:
ablating cardiac tissue on a first side superior to a right superior pulmonary vein and a left superior pulmonary vein in a transverse pericardial sinus;
ablating cardiac tissue on a second side lateral to a left superior pulmonary vein and a left inferior pulmonary vein; and
ablating cardiac tissue on a third side inferior to a left inferior pulmonary vein and a right inferior pulmonary vein in an oblique pericardial sinus.

22. The method of claim 21, wherein performing the second ablation comprises ablating cardiac tissue on a fourth side lateral to a right superior pulmonary vein and a right inferior pulmonary vein.

23. The method of claim 22, wherein performing the second ablation further comprises:
placing the ablation element held by the coupling element against a pericardial reflection extending between a right superior pulmonary vein and a superior vena cava; and
directing energy from the ablation element and to cardiac tissue through the pericardial reflection.

24. A method of forming a lesion in a wall of a heart by epicardial ablation, comprising:
positioning an ablation element partially around a plurality of pulmonary veins on an epicardial surface of a heart of a patient;
ablating a first section of epicardial tissue adjacent to the ablation element using the ablation element;
removing the ablation element from the patient;
inserting an endoscope supporting the same or a different ablation element into the patient; and
ablating a second section of epicardial tissue adjacent to the ablation element using the ablation element supported by the endoscope.

25. The method of claim 24, wherein the first section includes epicardial surfaces on three of four sides of four pulmonary veins, and the second section includes the remaining fourth side of four pulmonary veins.

26. An epicardial ablation system, comprising:
an endoscopic surgical instrument comprising an elongate shaft having a proximal end and a distal end, a lens element attached to the distal end of the elongate shaft, and a coupling element extending from the lens element; and
an ablation element, wherein the coupling element is configured to support the ablation element, and the ablation element is configured to form a lesion in a wall of heat by epicardial ablation.

27. The system of claim 26, wherein the ablation element is a flexible ablation element.

28. The system of claim 27, wherein the ablation element is a flexible microwave ablation element.

29. The system of claim 26, wherein a proximal end of the lens element defines an aperture configured to receive the distal end of the elongate shaft.

30. The system of claim 26, wherein the coupling element is attached to the lens element.
